(19) United States
(12) Patent Application Publication
(10) Pub. No.: US 2008/0234385 A1
(43) Pub. Date: Sep. 25, 2008

(54) Method For Inhibiting Tnf-Alpha

(75) Inventors: Gregory Oxenkrug, Newton, MA (US); Bertrand L. Jaber, South Boston, MA (US); Mary C. Perianayagam, Reading, MA (US)

Correspondence Address: DAVID S. RESNICK, NIXON PEABODY LLP, 100 SUMMER STREET, BOSTON, MA, 02110-2131, US

(73) Assignee: CARITAS ST. ELIZABETH'S MEDICAL CENTER, BOSTON, MA (US)

(21) Appl. No.: 11/997,484

(22) PCT Filed: Jul. 26, 2006
(86) PCT No.: PCT/US06/29127

§ 371 Date: Apr. 17, 2008

Related U.S. Application Data
(60) Provisional application No. 60/704,793, filed on Aug. 2, 2005.

(51) Int. Cl. A61K 31/165; C12N 5/06; A61P 19/00

(52) U.S. Cl. 514/622; 435/375

(57) ABSTRACT

The present invention encompasses methods for inhibiting TNF-alpha expression with N-substituted dopamine derivatives. In this method a cell is administered a pharmaceutically effective amount of N-acetyl dopamine derivatives or N-alkyldopamine derivatives and a pharmaceutically acceptable carrier for treating a cell, preferably in an animal or human suffering from overexpression or abundant TNF-alpha. The N-acetyldopamine derivative or N-alkyldopamine derivatives may be administered alone or in combination with N-acetylserotonin (NAS) or other compound to inhibit TNF-alpha expression. Also disclosed is a method of treating a TNF-alpha related disease and/or disorder using such a compound.

### CROSS REFERENCE TO RELATED APPLICATIONS

This application claims the benefit under 35 U.S.C. 119(e) of U.S. Provisional Patent Application Ser. No. 60/704,793, fled Aug. 2, 2005, the contents of which are herein incorporated by reference in their entirety.

### BACKGROUND OF THE INVENTION

Vertebrates achieve internal homeostasis during infection or injury by balancing the activities of pro-inflammatory and anti-inflammatory pathways. However, in many disease conditions, this internal homeostasis becomes out of balance. For example, endotoxin (lipopolysaccharide, LPS) produced by all Gram-negative bacteria activates macrophages to release cytokines that are potentially lethal (Tracey et al., 1986; Wang et al., 1999; Nathan, 1987; Dinarello, 1994).

Inflammation and other deleterious conditions (such as septic shock caused by endotoxin exposure) are often induced by pro-inflammatory cytokines, such as tumor necrosis factor-alpha (TNF-α; also known as cachectin). TNF-α, a mononuclear cytokine, is released by macrophages, monocytes and natural killer cells and plays a role in inflammatory and immunological events. TNF-α causes a broad spectrum of effects both in vitro and in vivo, including: (i) vascular thrombosis and tumor necrosis; (ii) inflammation; (iii) activation of macrophages and neutrophils; (iv) leukocytosis; (v) apoptosis; and (vi) shock. TNF-α has been associated with a variety of disease states including various forms of cancer, arthritis, psoriasis, endotoxic shock, sepsis, autoimmune diseases, infections, obesity, and cachexia. TNF-α also appears to play a role in the three factors contributing to body weight control: intake, expenditure, and storage of energy (Rothwell, Int. J. Obesity 17: S98-S101, 1993). In septicemia, increased endotoxin concentrations appear to raise TNF-α levels (Beutler et al. Science 229: 869-871, 1985).

TNF-α is a potent inducer of inflammation and many diseases and/or disorders related to expression of TNF-α cause serious morbidity and mortality for numerous individuals. For example, TNF-α related diseases or disorders include, but are not limited to, rheumatoid arthritis, juvenile rheumatoid arthritis, osteoarthritis, spondyloarthropathies, inflammatory bowel disease (including Crohn's disease and ulcerative colitis), chronic heart failure, systemic lupus erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, central nervous system injury, cancer (e.g., lung carcinomas, esophagus carcinoma, gastric adenocarcinoma, and prostate carcinoma), viral respiratory disease, and obesity. (See, e.g., Ogata H. et al Curr Pharm Des. 2003; 9(14): 1107-13; Moller D. R. et al J Intern Med. 2003; 253(1): 311-40; Taylor P. C. et al Curr Pharm Des. 2003; 9(14): 1095-106; Wilkinson N. et al Arch Dis Child. 2003; 88(3): 186-91; Nishimura F. et al J Periodontol. 2003; 74(1): 97-102; Weinberg J. M. et al Cutis. 2003; 71(1): 41-5; Burnham E. et al Crit Care Med. 2001; 29(3): 690-1; Sack M. et al Pharmacol Ther. 2002; 94(1-2): 123-35); Barnes P. J. et al Annu Rev Pharmacol Toxicol. 2002; 42:81-98; Mageed R. A. et al Lupus 2002; 11 (12): 850-5; Tsimberidou A. M. et al Expert Rev Anticancer Ther. 2002; 2(3): 277-86; Muller T. et al Curr Opin Investig Drugs. 2002; 3(12): 1763-7; Calandra T. et al Curr Clin Top Infect Dis. 2002; 22:1-23; Girolomoni G et al Curr Opin Investig Drugs. 2002; 3(11): 1590-5; Tutuncu Z. et al Clin Exp Rheumatol. 2002; 20(6 Suppl 28): S146-51; Braun J. et al Best Pract Res Clin Rheumatol. 2002; 16(4): 631-51; Barnes P. J. et al Novartis Found Symp. 2001; 234:255-67; discussion 267-72; Brady M. et al Baillieres Best Pract Res Clin Gastroenterol. 1999; 13(2): 265-89; Goldring M. B. et al Expert Opin Biol Ther. 2001; 1(5): 817-29; Mariette X. Rev Prat. 2003; 53(5): 507-11; Sharma R. et al Int J Cardiol. 2002; 85(1): 161-71; Wang C. X. et al Prog Neurobiol. 2002; 67(2): 161-72; Van Reeth K. et al Vet Immunol Immunopathol. 2002; 87(3-4): 161-8; Leonard B. E. et al Int J Dev Neurosci. 2001; 19(3): 305-12; and Hays S. J. et al Curr Pharm Des. 1998; 4(4): 335-48.

Attempts have been made to alter the course of such diseases and disorders by treating patients with TNF-α inhibitors, with varying degrees of success. For example, the TNF-α inhibitor dexanabinol provided protection against TNF-α mediated effects following traumatic brain injury (Shohami et al. J. Neuroimmun. 72: 169-77, 1997). Some improvement in Crohn's disease, Rheumatoid arthritis and psoriasis was afforded by treatment with anti-TNF-α antibodies (Neurath et al., Eur. J. Immun. 27: 1743-50, 1997). However, all of the currently available therapies have drawbacks such as toxicity, resistance, and low efficiency.

Thus, a method to inhibit TNF-α expression in vivo and in vitro is needed to inhibit, prevent, and/or treat various TNF-a mediated diseases and disorders.

### SUMMARY OF THE INVENTION

The present invention relates to a method for inhibiting TNF-alpha expression in vivo or in vitro. Thus, an aspect of this invention relates to a method of inhibiting expression of TNF-alpha in a subject in need thereof. The method includes administering to the subject a pharmaceutically effective amount of N-substituted dopamine derivatives and a pharmaceutically acceptable carrier for treating an animal or human suffering abnormal TNF-alpha related effects. N-substituted dopamine derivatives may be N-acetyldopamine derivatives or N-alkyldopamine derivatives. N-substituted dopamine derivatives may be administered alone or in combination with a pharmaceutically effective amount of N-acetylserotonin (NAS) or other compound to prevent or inhibit TNF-alpha expression. In a preferred embodiment, the N-substituted dopamine derivative is N-acetyldopamine.

According to the invention, N-substituted dopamine derivatives exhibit beneficial therapeutic properties and are useful in the treatment of TNF-alpha associated diseases or disorders. TNF-alpha related diseases and/or disorders may be induced by over-expression or increased production of TNF-alpha. Examples of TNF-alpha related disorders include, but are not limited to, rheumatoid arthritis, juvenile rheumatoid arthritis, osteoarthritis, spondyloarthropathies, inflammatory bowel disease (including Crohn's disease and ulcerative colitis), chronic heart failure, diabetes mellitus, systemic lupus, erythematosus, scleroderma, sarcoidosis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, central nervous system injury, lung carcinomas, esophagus carcinoma, gastric adenocarcinoma, prostate carcinoma, viral respiratory disease, and obesity. Alternatively, the disease or disorder may be multiple sclerosis, a burn, aging, metabolic syndrome (dyslipidemia, diabetes, hypertention) or the toxic effects of chemotherapy or radiation therapy.

The present invention provides for a method of inhibiting the expression of TNF-alpha in vivo or in vitro, comprising contacting a cell with, or administering to the subject, an effective amount of a compound of Formula (I), which is described fully below. In another embodiment, a method for treating a TNF-alpha related disease or disorder is described. In this method, a subject in need thereof is administered an effective amount of a compound of formula I.

The details of one or more embodiments of the invention are set forth in the accompanying drawings and the description below. Other features, objects, and advantages of the invention will be apparent from the description and drawings, and from the claims.

### DESCRIPTION OF THE FIGURES

FIG. 1: Effect of NAD, NMD, HMP and melatonin on LPS-stimulated TNF-α production. Differentiated THP-1 cells were co-incubated with endotoxin (10 ng/ml) and various concentrations of N-acetyldopamine (NAD), or N-methyldopamine (NMD) or 3-hydroxy-4-methoxydopamine (HMP) or melatonin (1-20 μM) for 24 hours. Cell supernatants were assayed for TNF-α by ELISA. Data are presented as mean ±SEM (N=6). P<0.01 for NAD vs all other agents at 1, 10 and 20 μM.

FIG. 2: Effect of Melatonin, NAS and NAD on LPS-stimulated TNF-alpha production. Differentiated THP-1 cells were co-incubated with endotoxin (10 ng/ml) and rising concentrations of melatonin, NAS or NAD (0-400 μM) for 24 hours. Cell supernatants were assayed for TNF-alpha by ELISA. *P=0.03 vs. control. Data are presented as mean±SEM (N=6).

### DETAILED DESCRIPTION OF THE INVENTION

This invention includes methods of inhibiting expression of TNF-alpha in vitro or in vivo. The present invention also includes methods of treating a TNF-alpha related disease and/or disorder with an effective amount of amount of N-substituted dopamine derivatives and a pharmaceutically acceptable carrier. The term “an effective amount” refers to the amount of the compound that is required to confer therapeutic effect in a subject. Effective amounts may vary, as recognized by those skilled in the art, depending on route of administration, excipient usage, and optional co-usage with another therapeutic agent. The term “treating” refers to administering a compound to a subject that has a TNF-alpha related disease or disorder, or has a symptom of the disease or disorder, or has a predisposition toward the disease or disorder, with the purpose to cure, prevent, ameliorate or inhibit the disease or disorder, or with the purpose to alleviate the symptoms of the TNF-alpha related disease or disorder.

N-substituted dopamine derivatives useful in the invention includes N-acetyldopamine derivatives or N-alkyldopamine derivatives. The N-substituted dopamine derivatives include compounds of Formula (I):

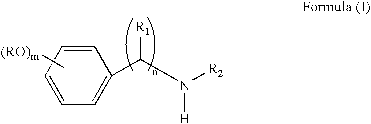

or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms.

As indicated in Formula (I), for the N-substituted dopamine derivatives used in the invention, R₂ is optionally substituted C₁-C₆ alkyl, or —C(═O)R₃. In the preferred compounds, R₂ is —CH₃ or —C(═O)R₃.

The term “alkyl,” unless otherwise modified, refers to straight chain and branched alkyls as well as cyclic groups, although of course cyclic groups will comprise at least three carbon ring members.

Preferred alkyl groups of compounds of Formula (I) have from one to 6 carbon atom, more preferably one to six atoms, and even more preferably 1, 2, or 3 atoms. The alkenyl and alkynyl groups of compounds of the invention have one or more unsaturated linkages and typically from 2 to about 6 carbon atoms, and more preferably 2 to about 4 carbon atoms. The terms “alkenyl” and “alkynyl” as used herein refer to both straight chain or branched cyclic groups and noncyclic groups, although straight or branched noncyclic groups are generally more preferred.

The term “substituted” mean that the substitution with common substituents known in the art. Examples of such substituents include hydroxyl groups, thiol groups, halogens, amino groups, nitro groups, sulphate groups, phosphate groups, carboxylic acid groups, esters, amides, and the like.

Preferred alkoxy groups of compounds of the invention include groups having one or more oxygen linkages and from 1 to about 6 carbon atoms, and more preferably from 1 to about 4 carbon atoms. The alkoxy groups may be straight chain or branched groups, saturated or unsaturated.

Preferred alkylthio groups of compounds of the invention include those groups having one or more thioether linkages and from 1 to about 6 carbon atoms, and more preferably from 1 to about 4 carbon atoms. The alkylthio groups may be straight chain or branched groups, saturated or unsaturated as well as cyclic groups, although of course cyclic groups will comprise at least three carbon ring members.

Preferred alkylsulfinyl groups of compounds of the invention include those groups having one or more sulfoxide (SO) groups and from 1 to about 6 carbon atoms, and more preferably from 1 to about 4 carbon atoms. The alkylsulfinyl groups may be straight chain or branched groups, saturated or unsaturated as well as cyclic groups, although of course cyclic groups will comprise at least three carbon ring members.

Preferred alkylsulfonyl groups of compounds of the invention include those groups having one or more sulfonyl (SO₂) groups and from 1 to about 6 carbon atoms, and more preferably from 1 to about 4 carbon atoms. The alkylsulfonyl groups may be straight chain or branched groups, saturated or unsaturated as well as cyclic groups, although of course cyclic groups will comprise at least three carbon ring members.

Preferred aminoalkyl groups include those groups having one or more primary, secondary and/or tertiary amine groups, and from 1 to about 6 carbon atoms, and more preferably 1 to about 4 carbon atoms. Secondary and tertiary amine groups are generally more preferred than primary amine moieties. The aminoalkyl groups may be straight chain or branched groups, saturated or unsaturated as well as cyclic groups, although of course cyclic groups will comprise at least three carbon ring members.

It should be understood that alkoxy, alkylthio, alkylsulfinyl, alkylsulfonyl and aminoalkyl substituent groups described above include groups where a hetero atom is directly bonded to a ring system, such as a carbocyclic aryl group or a heterocyclic group, as well as groups where a hetero atom of the group is spaced from such ring system by an alkylene linkage, e.g. of 1 to about 4 carbon atoms.

Without wishing to be bound by theory, compounds of the invention that contain an alkylsulfinyl and/or alkylsulfonyl group, may be, in effect, “pro-drugs” wherein after administration of the compound to a subject the sulfinyl or sulfonyl group(s) are metabolized (reduced) in vivo to the corresponding sulfide moiety.

As is known in the art, pharmaceutically acceptable salts of the N-substituted dopamine derivatives may also be used in the methods of invention. Suitable pharmaceutically acceptable salts include acid addition salts such as those prepared from the following acids: hydrochloric, hydrobromic, hydroiodic, sulfuric, nitric, phosphoric, maleic, salicylic, p-toluenesulfonic, tartaric, citric, acetic, methanesulfonic, formic, succinic, naphthalene-2-sulfonic, isethionic, lactobionic and benzenesulfonic.

Suitable heteroaromatic groups of compounds of the invention contain one or more N, O or S atoms and include, e.g., coumarinyl including 8-coumarinyl, quinolinyl including 8-quinolinyl, pyridyl, pyrazinyl, pyrimidyl, furyl, pyrrolyl, thienyl, thiazolyl, oxazolyl, oxidizolyl, triazole, imidazolyl, indolyl, benzofuranyl and benzothiazol.

Suitable hetefoalicyclic groups of compounds of the invention contain one or more N, O or S atoms and include, e.g., tetrahydrofuranyl, thienyl, tetrahydropyranyl, piperidinyl, morpholino and pyrrolindinyl groups.

Suitable carbocyclic aryl groups of compounds of the invention include single and multiple ring compounds, including multiple ring compounds that contain separate and/or fused aryl groups. Typical carbocyclic aryl groups of compounds of the invention contain 1 to 3 separate or fused rings and from 6 to about 18 carbon ring atoms. Specifically preferred carbocyclic aryl groups include phenyl; naphthyl including 1-naphthyl and 2-naphthyl; biphenyl; phenanthryl; anthracyl; and acenaphthyl. Substituted carbocyclic groups are particularly suitable including substituted phenyl, such as 2-substituted phenyl, 3-substituted phenyl, 4-substituted phenyl, 2,3-substituted phenyl, 2,5-substituted phenyl, 2,3,5-substituted and 2,4,5-substituted phenyl; and substituted naphthyl, including naphthyl substituted at the 5, 6 and/or 7 positions. Preferred substituents of such substituted carbocyclic groups are identified below.

Suitable aralkyl groups of compounds of the invention include single and multiple ring compounds, including multiple ring compounds that contain separate and/or fused aryl groups. Typical aralkyl groups contain alkyl chain as discussed above and 1 to 3 separate or fused rings and from 6 to about 18 carbon ring atoms. Preferred aralkyl groups include benzyl and methylenenaphthyl (—CH₂ -naphthyl).

Specifically preferred compounds of the invention include N-acetyldopamine, N-chloroacetyldopamine, N-methyldopamine (deoxyepinephrine) or N-acetyl-m-tyramine to treat TNF-alpha disease or disorder or inhibit expression of TNF-alpha, according to the invention. N-acetyldopamine, N-chloroacetyldopamine, N-methyldopamine (deoxyepinephrime), N-acetyl-m-tyramine, and NAS may be prepared by those methods known in the art or purchased commercially.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines. The precise dosage will naturally depend on a number of clinical factors, for example, the age of the recipient, the route of administration and the condition under treatment, its severity, and whether one or a combination of compounds is administered. The desired daily dose is preferably given as two or three or more subdoses administered at appropriate intervals during the day.

According to the methods of the invention, the compounds of Formula (I) may be administered for the treatment of each of the disorders stated herein above, in the dosage range of 0.01 mg/kg to 500 mg/kg of human body weight per day, preferably about 0.1 mg/kg to about 50 mg/kg of human body weight per day and optimally about 10 mg/kg of human body weight per days

Suitable effective dose of N-acetylserotonin in the combination of the compound of Formula (I) will be in the range of from 0.01 to 100 milligrams per kilogram of bodyweight of recipient per day, preferably in the range of from 0.01 to 20 milligrams per kilogram bodyweight of recipient per day, more preferably in the range of 0.05 to 4 milligrams per kilogram bodyweight of recipient per day.

While it is possible to administer N-acetyldopamine, N-chloroacetyldopamine, N-acetyl-m-tyramine, or NAS as a raw chemical, it is highly desirable to administer it in the form of a pharmaceutical composition comprising N-acetyldopamine, N-chloroacetyldopamine, N-acetyl-m-tyramine, or NAS together with an acceptable carrier therefor; the carrier should be acceptable in the sense of being compatible with the other ingredients and not deleterious to the recipient thereof. The compositions may be adapted for oral, transdermal, parenteral or rectal administration. Oral administration is preferred. In case of septic shock and SIRS the parenteral administration is preferred.

The pharmaceutical compositions may conveniently be presented in unit dosage form and may be prepared by any methods well known in the art of pharmacy. Preferred unit dosage formulations are those containing a daily subdose, as herein above recited, or an appropriate fraction thereof, of the active ingredient. Such methods include the step of bringing into association the active ingredient with the carrier which may comprise one or more accessory ingredients. In general the pharmaceutical compositions are prepared by uniformly and intimately bringing into association the active ingredient with liquid carriers or finely divided solid carriers or both, and then, if necessary, shaping or encapsulating the product.

Pharmaceutical compositions suitable for oral administration may be presented in discrete units such as capsules, cachets or tablets each containing a predetermined amount of active ingredient; as a powder or granules; as a solution or a suspension in an aqueous liquid or a non-aqueous liquid; or as an oil-in-water liquid emulsion or a water-in-oil liquid emulsion.

A tablet may be made by compression or molding, optionally with one or more accessory ingredients. Compressed tablets may be prepared by compressing in a suitable machine the active ingredient in a free-flowing form such as a powder of granules, optionally mixed with a binder, lubricant, inert diluent, and surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

The pharmaceutical compositions of the invention may be formulated into other forms depending upon the desired route of administration as is know in the art. Pharmaceutical compositions suitable for rectal administration may be presented as a suppository with the usual carriers such as cocoa butter. Pharmaceuticals compositions suitable for parenteral administration include aqueous sterile injection solutions which may contain antioxidants, buffers, bacteriostats and solutes which renders the parenteral composition isotonic with the blood of the intended recipient; and aqueous and non-aqueous sterile suspensions which may include suspending agents and thickening agents. The parenteral compositions may be presented in unit doses or multidose containers, for example, sealed ampoules and vials, and may be stored in a freeze dried (lyophilized) condition requiring only the addition of the sterile liquid carrier, for example, PEG 400: ethanol mixtures, immediately prior to use. Extemporaneous injection solutions and suspensions may be prepared from sterile powders, granules and tablets of the kind previously described.

It should be understood that in addition to the ingredients particularly mentioned above, the pharmaceutical compositions of this invention may include other agents conventional in the art having regard to the type of composition in question, for example, those suitable for oral administration may include flavoring agents.

According to the invention the N-substituted dopamine derivatives may be administered alone or in combination with other therapeutically effective agents. Use of N-substituted dopamine derivatives in combination with other drugs used presently in inhibiting TNF-alpha such as, for example, NAS, may reduce the concentration of drug needed for successful treatment, thereby alleviating some of the major side effects observed with higher doses. Furthermore, the period observed between administering the drugs and any observed therapeutic indications may be diminished.

As used herein and throughout, “NAS” is meant to include N-acetyl-serotonin and pharmaceutically acceptable salt or prodrug form thereof. “NAD” is meant to include N-acetyldopamine and pharmaceutically acceptable salt or prodrug form thereof.

### EXAMPLES

### Experimental Methods

### THP-1 Cell Culture

The human promonocytic THP-1 cell line (American Type Culture Collection, Manassas, Va.) was maintained in suspension culture at 0.25×10⁶ cells/ml in RPMI-1640 medium supplemented with 2 mM L-glutamine, 10% fetal bovine serum and 0.05 mM 2-mercaptoethanol in a humidified atmosphere of 5% CO₂ in air at 37° C. Differentiation to monocytes (0.25×10⁶ cells/ml) was induced by incubating cells with trans-retinoic acid (1 μM), 1.25 dihydroxy cholecalciferol (0.1 μM) and interferon-γ (0.01 mg/l) (Sigma, St. Louis, Mo.). After 3 days, cells were detached using trypsin-EDTA, washed in PBS, and 1×10⁶ cells were used for each experimental condition.

### Cytokine Production by THP-1-Derived Monocytes

Differentiated THP-1 cells were co-incubated with 10 ng/ml of purified Escherichia coli endotoxin (serotype O55: B5, Sigma Chemical Co.) and rising concentrations (0-400 μM) of either melatonin, N-acetyldopamine (NAD) or N-methyldopamine (NMD) or 3-hydroxy-4-methoxy-dopamine (HMP) (Sigma) in RPMI-1640 cell culture medium at 37° C. in a humidified atmosphere supplemented with 5% CO₂. After 24-hour incubation, cell supematants were harvested, and TNF-α was measured by a sandwich enzyme linked immunosorbent assay (ELISA), according to the manufacturer's instructions (Quantikine®, R & D systems, Minneapolis, Minn., USA). The lower limit of detection for TNF-α was 1.6 pg/ml. The average intra-assay coefficient of variation for TNF-α was 5%.

### Statistical Analysis

Statistical analysis was performed using the Statistical Package for Social Sciences version 10.0 (SPSS, Chicago, Ill.). Comparisons between groups were made by ranked non-parametric Kruskall Wallis analysis of variance (ANOVA) or Friedman test, and two-tailed Mann Whitney test (unpaired and paired) for continuous variables. Results are expressed as means±standard error of the mean (SEM). Differences were considered statistically significant at P<0.05.

### Results

### Effect of NAD, NMD, HMP and Melatonin, on LPS-Stimulated TNF-α Production

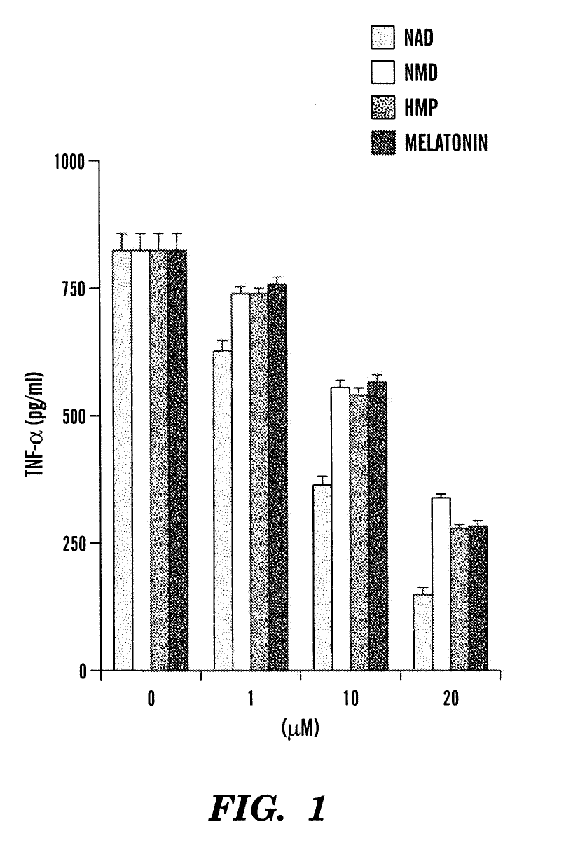

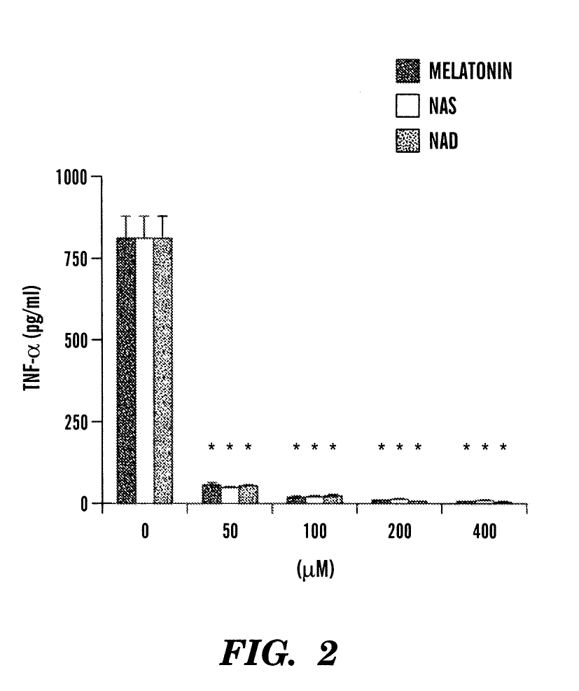

Incubation of THP-1 derived monocytes with rising concentrations of melatonin, NAS and NAD resulted in a marked decrease in LPS-stimulated TNF-α production, which was dose dependent. Indeed, as shown in FIG. 1 and FIG. 2, compared with control conditions, melatonin, NAS and NAD significantly inhibited TNF-α production by 96 to 98% (P=0.03).

### Discussion

In the present study, we demonstrated that NAD and its derivatives, NMD and HMP suppress LPS-stimulated TNF-α synthesis by monocytic cells in a dose-dependent manner. Low-dose NAD was more potent in blocking TNF-α production compared with NMD, HMP and melatonin, the powerful endogenous inhibitor of TNF-α production.

Our study is the first to demonstrate the protective effect of NAD and its derivatives on LPS-induced TNF-α production.

### REFERENCES

1. Faure, E., L. Thomas, H. Xu, A. Medvedev, O. Equils & M. Arditi. 2001. Bacterial lipopolysaccharide and IFN-gamma induce Toll-like receptor 2 and Toll-like receptor 4 expression in human endothelial cells: role of NF-kappa B activation. J. Immunol. 166: 2018-2024.
2. Sewerynek, E., D. Melchiorri, L. Chen & R. J. Reiter. 1995. Melatonin reduces both basal and bacterial lipopolysaccharide-induced lipid peroxidation in vitro. Free Rad. Biol. Med. 19: 903-909.
3. Maestroni, G. J. 1996. Melatonin as a therapeutic agent in experimental endotoxic shock. J. Pineal Res. 20: 84-89.
4. Requintina, P. J. & Oxenkrug, G. F. 2003. Differential effects of lipopolysaccharide on lipid peroxidation in F344N, SHR rats and BALS/c mice, and protection of melatonin and NAS against its toxicity. Annals. N.Y. Acad. Sci. 993: 325-333.
5. Gitto, E., C. Romeo, R. J. Reiter, P. Impellizzeri, S. Pesce, M. Basile, P. Antonuccio, G. Trimarchi, C. Gentile, I. Barberi & B. Zuccarello. 2004. Melatonin reduces oxidative stress in surgical neonates. J. Pediatr. Surg. 39: 184-189.
6. Young, I. M., R. M. Leone, P. Francis, P. Stovell & R. E. Silman. 1985. Melatonin is metabolized to N-acetyl serotonin and 6-hydroxymelatonin in man. J. Clin. Endocrinol. Metab. 60: 114-119.
7. Klemm, P., J. Ostrowski, T. Morath, C. Gruber, P. A. Martorana & R. Henning. 1993. N-Acetylserotonin prevents the hypotension induced by bacterial lipopolysaccharides in the rat. Euro. J. Pharmacol. 250: R9-R10.
8. Sacco, S., L. Aquilini, P. Ghezzi, M. Pinza & A. Guglielmotti. 1998. Mechanism of the inhibitory effect of melatonin on tumor necrosis factor production in vivo and in vitro. Euro. J. Pharmacol. 343: 249-255.
9. Klemm, P., M. Hecker, H. Stockhausen, C. C. Wu & C. Thiemermann. 1995. Inhibition by N-acetyl-5-hydroxytryptamine of nitric oxide synthase expression in cultured cells and in the anaesthetized rat. Bri. J. Pharmacol. 115: 1175-1181.
10. Reiter, R. J., L. Tang, J. J. Garcia & A. Munoz-Hoyos. 1997. Pharmacological action of melatonin in oxygen radical pathophysiology. Life Sci. 60: 2255-2271.
11. Pieri, C., M. Marra, R. Gaspar & S. Damjanovich. 1996. Melatonin protects LDL from oxidation but does not prevent the apolipoprotein derivatization. Biophy. Res. Commun. 222: 256-260.
12. Melchiorri, D., R. J. Reiter, A. M. Attia, M. Hara, A. Burgos & G. Nistico. 1995. Potent protective effect of melatonin on in vivo paraquat-induced oxidative damage in rats. Life Sci. 56: 83-89.
13. Manev, H., C. M. Cagnoli, A. Kharlamov, C. Atabay & E. Kharlamov. 1995. In vitro and in vivo neuroprotection with melatonin against the toxicity of singlet oxygens. Soc. Neurosci. Abstr.21: 1518.
14. Sewerynek, E., D. Melchiorri, R. J. Reiter, G. G. Ortiz & A. Lewinski. 1995. Lipopolysaccharide-induced hepatotoxicity is inhibited by the antioxidant melatonin. Eur. J. Pharmacol. 293: 327-334.
15. Melchiorri, D., E. Sewerynek, R. J. Reiter, G. G. Ortiz, B. Poeggeler & G. Nistico. 1997. Suppressive effect of melatonin administration on ethanol-induced gastroduodenal injury in rats in vivo. Br. J. Pharmacol. 121: 264-270.
16. Ben-Nathan, D., G. J. Maestroni, S. Lustig & A. Conti, 1995. Protective effects of melatonin in mice infected with encephalitis viruses. Arch. Virol. 140: 223-230.
17. Thony, B, G. Auerbach & N. Blau. 2000. Tetrahydrobiopterin biosynthesis, regeneration and functions. Biochem. J. 347 (Pt 1): 1-16.
18. Dong W G, Mei Q, Yu J P, Xu J M, Xiang L, Xu Y. Effects of melatonin on the expression of iNOS and COX-2 in rat models of colitis. World J Gastroenterol. June 2003;9(6):1307-11.
19. Werner, E. R., G. Werner-Felmayer, D. Fuchs, A. Hausen, G. Reibnegger, J. J. Yim & H. Wachter. 1991. Impact of tumour necrosis factor-alpha and interferon-gamma on tetrahydrobiopterin synthesis in murine fibroblasts and macrophages. Biochem. J. 280 (Pt 3): 709-714.

The references cited throughout the application are incorporated herein by reference in their entirety.

What is claimed is:

1. A method of inhibiting the expression of TNF-alpha in a cell in need thereof, comprising contacting the cell with an effective amount of a compound of Formula (I): 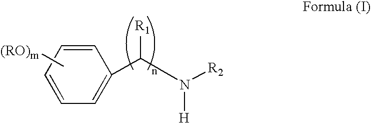
or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl, or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms.

2. The method of claim 1, wherein;
R is independently hydrogen, or optionally substituted C₁-C₄ alkyl;
m is 1 or 2;
R₁ is each independently hydrogen, halogen, or optionally substituted C₁-C₄ alkyl;
n is 1, 2, or 3;
R₂ is —CH₃, or —C(═O)R₃;
R₃ is independently optionally substituted C₁-C₄ alkyl; optionally substituted C₂-C₆ alkenyl or optionally substituted C₃-C₄ alkynyl.

3. The method of claim 1, wherein the compound of Formula (I) is selected from the group consisting of N-acetyldopamine, N-chloroacetyldopamine, N-methyldopamine, and N-acetyl-m-tyramine.

4. The method of claim 3, wherein the compound of Formula (I) is N-acetyldopamine.

5. The method of claim 3, wherein the compound of Formula (I) is N-methyldopamine.

6. The method of claim 3, wherein the compound of Formula (I) is N-chloroacetyldopamine.

7. The method of claim 1, wherein the cell is in culture.

8. The method of claim 1, wherein the cell is in a host.

9. A method for treating a TNF-alpha related disease and/or disorder, comprising administering to a subject in need thereof an effective amount of a compound of Formula (I): 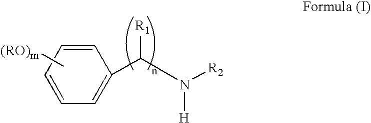
or a pharmaceutically acceptable salt or prodrug form thereof,
wherein
R is independently hydrogen, optionally substituted C₁-C₆ alkyl, optionally substituted C₂-C₆ alkenyl, and optionally substituted C₂-C₆ alkynyl;
m is 0, 1, 2, 3, 4 or 5;
R₁ is independently hydrogen, halogen, optionally substituted C₁-C₆ alkyl, optionally substituted C₂-C₆ alkenyl, optionally substituted C₂-C₆ alkynyl, optionally substituted C₁-C₆ alkoxy, optionally substituted C₁-C₆ alkylthio, optionally substituted C₁-C₆ alkylsulfinyl, optionally substituted C₁-C₆ alkylsulfonyl, optionally substituted C₁-C₆ aminoalkyl, optionally substituted carbocyclic aryl, or optionally substituted aralkyl;
n is 1, 2, or 3;
R₂ is optionally substituted C₁-C₆ alkyl, or —C(═O)R₃;
R₃ is independently optionally substituted C₁-C₆ alkyl, optionally substituted C₂-C₆ alkenyl, optionally substituted C₂-C₆ alkynyl, optionally substituted or unsubstituted carbocyclic aryl, or an optionally substituted heteroaromatic or heteroalicyclic group having from 1 to 3 rings, 3 to about 8 ring members in each ring and 1 to about 3 hetero atoms.

10. The method of claim 9, wherein; R is independently hydrogen, or optionally substituted C₁-C₄ alkyl;
m is 1 or 2;
R₁ is each independently hydrogen, halogen, or optionally substituted C₁-C₄ alkyl;
n is 1, 2, or 3;
R₂ is —CH₃, or —C(═O)R₃;
R₃ is independently optionally substituted C₁-C₄ alkyl; optionally substituted C₂-C₆ alkenyl or optionally substituted C₃-C₄ alkynyl.

11. The method of claim 9, wherein the compound of Formula (I) is selected from the group consisting of N-acetyldopamine, N-chloroacetyldopamine, N-methyldopamine, and N-acetyl-m-tyramine.

12. The method of claim 11, wherein the compound of Formula (I) is N-acetyldopamine.

13. The method of claim 11, wherein the compound of Formula (I) is N-methyldopamine.

14. The method of claim 11, wherein the compound of Formula (I) is N-chloroacetyldopamine.

15. The method of claim 9, wherein the TNF-alpha related disease and/or disorder is selected from the group consisting of rheumatoid arthritis, juvenile rheumatoid arthritis, osteoarthritis, spondyloarthropathies, inflammatory bowel disease, chronic heart failure, diabetes mellitus, systemic lupus, erythematosus, scleroderma, sarcoidosis, Crohn's Disease, psoriasis, polymyositis/dermatomyositis, psoriasis, multiple myeloma, myelodysplastic syndrome, acute myelogenous leukemia, Parkinson's disease, AIDS dementia complex, Alzheimer's disease, depression, sepsis, pyoderma gangrenosum, hematosepsis, septic shock, Behcet's syndrome, graft-versus-host disease, uveitis, Wegener's granulomatosis, Sjogren's syndrome, chronic obstructive pulmonary disease, asthma, acute pancreatitis, periodontal disease, cachexia, central nervous system injury, viral respiratory disease, and obesity.

16. The method of claim 9, wherein the TNF-alpha related disease and/or disorder is rheumatoid arthritis.

17. The method of claim 9, wherein the TNF-alpha related disease and/or disorder is Crohn's disease.

18. The method of claim 9, wherein the TNF-alpha related disease and/or disorder is sepsis.
